(19) United States
(12) Patent Application Publication
(10) Pub. No.: US 2011/0081697 A1
(43) Pub. Date: Apr. 7, 2011

(54) Progressive Fermentation of Lignocellulosic Biomass

(76) Inventor: Chaogang Liu, Hanover, NH (US)

(21) Appl. No.: 12/680,311

(22) PCT Filed: Sep. 29, 2008
(86) PCT No.: PCT/US08/78136

§ 371 Date: Dec. 15, 2010

Related U.S. Application Data
(60) Provisional application No. 60/975,660, filed on Sep. 27, 2007.

(51) Int. Cl. C12P 7/14

(52) U.S. Cl. 435/162

(57) ABSTRACT

Provided are methods for the efficient and cost-reduced production of ethanol or other fermentation products or both from cellulosic biomass, which methods exploit the optimal features of yeasts, fungi, and bacteria while simultaneously minimizing their limitations. For example, one aspect of the present invention relates to methods of producing ethanol or other fermentation products or both from lignocellulosic biomass via progressive fermentation using in series or parallel two or more of yeast, fungus, and bacteria.

### RELATED APPLICATIONS

This application claims the benefit of U.S. Provisional Patent Application Ser. No. 60/975,660, filed Sep. 27, 2007; the entire contents of which is incorporated herein by reference.

### BACKGROUND OF THE INVENTION

Energy conversion, utilization and access underlie many of the great challenges of our era, including those associated with sustainability, environmental quality, security, and poverty. Emerging technologies are required to respond to these challenges, and, as one of the most powerful of these technologies, biotechnology can give rise to important new energy conversion processes.

Plant biomass and derivatives thereof are a resource for the biological conversion of energy to forms useful to humanity. Among forms of plant biomass, lignocellulosic biomass (‘biomass’) is particularly well-suited for energy applications because of its large-scale availability, low cost, and environmentally benign production. In particular, many energy production and utilization cycles based on cellulosic biomass have near-zero greenhouse gas emissions on a life-cycle basis. The primary obstacle impeding the more widespread production of energy from biomass feedstocks is the general absence of low-cost technology for overcoming the recalcitrance of these materials.

Lignocellulosic biomass contains carbohydrate fractions (e.g., cellulose and hemicellulose) that can be converted into ethanol. The production of ethanol from biomass typically involves the breakdown or hydrolysis of lignocellulose-containing materials into disaccharides and, ultimately, monosaccharides. Under anaerobic conditions (no available oxygen), fermentation occurs in which the degradation products of organic compounds serve as hydrogen donors and acceptors. Excess NADH from glycolysis is oxidized in reactions involving the reduction of organic substrates to products, such as lactate and ethanol. In addition, ATP is regenerated from the production of organic acids, such as acetate, in a process known as substrate level phosphorylation. Therefore, the fermentation products of glycolysis and pyruvate metabolism include a variety of organic acids, alcohols and CO₂.

The majority of facultatively anaerobic bacteria do not produce high yields of ethanol under either aerobic or anaerobic conditions. Most faculatative anaerobes metabolize pyruvate aerobically via pyruvate dehydrogenase (PDH) and the tricarboxylic acid cycle (TCA). Under anaerobic conditions, the main energy pathway for the metabolism of pyruvate is via the pyruvate-formate-lyase (PFL) pathway to give formate and acetyl-CoA. Acetyl-CoA is then converted to acetate, via phosphotransacetylase (PTA) and acetate kinase (AK) with the co-production of ATP, or reduced to ethanol via acetalaldehyde dehydrogenase (AcDH) and alcohol dehydrogenase (ADH). In order to maintain a balance of reducing equivalents, excess NADH produced from glycolysis is re-oxidized to NAD⁺ by lactate dehydrogenase (LDH) during the reduction of pyravate to lactate. NADH can also be re-oxidized by AcDH and ADH during the reduction of acetyl-CoA to ethanol but this is a minor reaction in cells with a functional LDH. Theoretical yields of ethanol, therefore, are not achieved because most acetyl CoA is converted to acetate to regenerate ATP and excess NADH produced during glycolysis is oxidized by LDH.

Ethanologenic organisms, such as Zymomonas mobilis, Zymobacter palmae, Acetobacter pasteurianus, and Sarcina ventriculi, and some yeasts (e.g., Saccharomyces cerevisiae), are capable of a second type of anaerobic fermentation, commonly referred to as alcoholic fermentation, in which pyruvate is metabolized to acetaldehyde and CO₂ by pyruvate decarboxylase (PDC). Acetaldehyde is then reduced to ethanol by ADH regenerating NAD⁺. Alcoholic fermentation results in the metabolism of one molecule of glucose to two molecules of ethanol and two molecules of CO₂.

Biological conversion of cellulosics to ethanol for use as an alternative fuel has a number of benefits; however, the high processing costs still challenge the commercialization of this technology. There are several processing options to produce ethanol from cellulosic biomass. Among them, simultaneous saccharification and fermentation (SSF) is an attractive option because it provides several unique advantages. By combining enzymatic hydrolysis and fermentation in one reactor, SSF significantly reduces capital investment and operating costs and decreases production of inhibiting products.

Yeast is widely used in the ethanol-production industry for its advantages in ethanol titer, inhibitor tolerance, and hardiness; however, yeast can only ferment hexoses, such as glucose. Economic analyses show that simultaneous conversion of all cellulose and hemicellulose sugars (e.g., glucose, xylose, galactose, arabinose, and mannose) into ethanol is the key to making the biomass-to-ethanol process economically feasible. While there is interest in developing pentose-fermentative yeasts, work is also being done with bacteria that are naturally capable of metabolizing all sugars to produce ethanol, organic acids, and other byproducts. Zymomonas and E. coli have been shown to be successfully engineered to produce ethanol as the only product. Similar to most yeasts, however, the optimal temperatures for the growth and fermentation for methophilic bacteria (<40° C.) is not an optimal match for the enzymes that are used in the process (50° C.). Accordingly, thermophilic anaerobic bacteria, such as T. sacch ALK2, that can grow at temperatures of up to 60° C. are better suited candidates for converting cellulosic biomass to ethanol via SSF. In addition, thermophilic bacteria produce hemicellulases concurrently that can enhance cellulose conversion with reduced enzyme loadings in the SSF process. However, most thermophilic anaerobic bacteria have a low tolerance to inhibitors, such as acetate, furfural, HMF, and phenolics, which are commonly present in pretreated biomass or hydrolysates. A number of methods are available for removal of toxics, including physical, chemical, and biochemical detoxification approaches, but none of these methods is economical. Moreover, anaerobic operation is expensive, particularly for the production of commodity chemicals.

### SUMMARY OF THE INVENTION

It is an object of the invention to provide a novel method or process that combines the optimal features of yeasts, fungi, and bacteria while, at the same time, overcoming their limitations for the efficient and cost-reduced production of ethanol from cellulosic biomass. Specific objects of the invention include, but are not limited to, the removal of oxygen and inhibitors, e.g., byproducts, by yeast or fungus, and the utilization of yeast or fungal biomass as a nitrogen source to enhance the subsequent fermentation with thermophilic bacteria for a high ethanol yield and productivity.

Aspects of the present invention relate to methods of producing ethanol and other fermentation products, from lignocellulosic biomass by progressive fermentation using yeast, fungus, and bacteria. The methodology described herein utilizes certain inherent properties and advantages of yeasts, including, for example, their robust ethanol titer, high inhibitor tolerance, and hardiness. Although yeasts grow in both aerobic and anaerobic environments, yeasts ferment only hexoses and grow in moderate temperatures which are not optimal characteristics for SSF. Some thermophilic bacteria, e.g., T. sacch, have been found to be able to convert all sugars derived from hemicellulose and cellulose to ethanol with a high ethanol yield and productivity; however, they can only grow in a strictly anaerobic environment that makes the fermentation operation complex and expensive. In addition, thermophilic bacteria are weakly resistant to inhibitors, such as acetic acid, furfural, HMF, and phenolics, that often make the fermentation of substrates very slow or unsuccessful.

Accordingly, in one aspect of the invention, progressive fermentation with yeast or fungi and thermophilic bacteria can combine the positive features of yeast, fungus, and thermophilic bacteria, realizing high sugar conversion, high ethanol yield, increased productivity, and low operation costs. It is further an object of the invention that yeast and fungi may be combined in the methods of the invention.

According to one embodiment, the invention provides a method for processing lignocellulosic material, comprising the steps of: placing a sample of lignocellulosic material in a reactor; adding to the reactor a yeast or fungus at a first temperature and a first pH to carry out a first fermentation and give a first mixture; adjusting the temperature and pH to autolyze the yeast or fungal cells in the broth to give a second mixture; adding to the second mixture a thermophilic microorganism and at least one enzyme at a third temperature and a third pH to give a third mixture; and allowing the third mixture to age for a period of time to give a fourth mixture; wherein said fourth mixture comprises a liquid product and a solid product; and said liquid product comprises ethanol.

In certain embodiments, oxygen, inhibitors (such as acetic acid, furfural, HMF, phenolics, and others), hemicellulose sugars (pentoses and hexoses) in the medium are completely or partially removed by fermentation with yeast or fungus, followed by fermentation with bacteria, thereby converting all hemicellulose sugars and cellulose into ethanol or other fermentation products, such as organic acids. Moreover, the presence of yeast or fungus in the methods of the invention will be beneficial to subsequent fermentation with thermophilic bacteria. As such, the autolyzed yeast or fungal cells at elevated temperatures and pH provide an excellent nutrient for bacterial growth. In addition, the enzymes released during autolysis are supplemental to the enzymes necessarily added in subsequent enzymatic hydrolysis and fermentation. Accordingly, the methods described herein may simplify the fermentation process, reduce the costs for the medium, enzymes and operations, and achieve high ethanol yield and productivity, leading to economically feasible production of ethanol and other chemicals, including organic acids from cellulosic biomass.

In one aspect of the invention, at least one enzyme may be added at any point during the process. Such enzymes may include, for example, a cellulolytic enzyme, e.g., cellulase, endoglucanase, cellobiohydrolase, and beta-glucosidase. In another embodiment, the method further comprises treating the lignocellulosic material with an effective amount of at least one enzyme, including hemicellulase, esterase, protease, laccase, peroxidase, or a mixture thereof. In yet another embodiment, a combination of enzymes may be used in a method of the invention.

The methods of the present invention may further comprise other processes known in the art, including, but not limited to, pretreatment and consolidated bioprocessing of the lignocellulosic material, thereby resulting in fewer degradation products and an overall higher ethanol yield. In one embodiment, lignocellulosic material is pretreated and stripped of easy to hydrolyze material. In certain other embodiments, it may be desirable to perform such processes at any point during the process.

In another aspect, it may also be advantageous to remove various components of the mixture, such as sugars, e.g., pentoses or hexoses, during the methods of the invention. In yet another aspect, ethanol may be readily removed at any point during the process using conventional methods.

In still another aspect, in addition to ethanol, other fermentation products (e.g., commodity and specialty chemicals) can be produced from lignocellulose, including xylose, acetone, acetate, glycine, lysine, organic acids (e.g., lactic acid), 1,3-propanediol, butanediol, glycerol, ethylene glycol, furfural, polyhydroxyalkanoates, cis,cis-muconic acid, and animal feed. In another aspect, such fermentation products may be removed at any point during the process using conventional methods.

As noted above, the bacteria used in the methods of the invention are thermophilic microorganisms. In another embodiment, the thermophilic bacteria are of the genera Thermoanaerobacterium or Thermoanaerobacter. In yet another embodiment, the bacteria are cellulolytic, xylanolytic thermophilic anaerobes.

Hemicellulases are expensive, and they are required enzymes in the cellulosic ethanol process. However, hemicellulases can be produced effectively and inexpensively based on the processes described herein. Accordingly, in one aspect, the invention requires removal of the soluble fraction from pretreated substrates with hot water, thereby increasing cellulose digestibility at reduced enzyme loadings. In another embodiment, the process described herein provides enhanced SSF of the solids and fermentability of the hydrolyzates for the partial removal of lignin and inhibitors.

In certain other embodiments, the invention features a soluble hemicellulose fraction from which pretreated substrates may be separated by hot washing and used as a carbon source to produce hemicellulases by fungi, such as T. reesei Rut 30. In one aspect, the entire broth comprises fungal cells and produces enzymes that are used for subsequent enzymatic hydrolysis and fermentation. By combining the fungi cells and the produced enzymes to perform enzymatic hydrolysis and fermentation, the enzymes work more efficiently. In another embodiment, a soluble hemicellulose fraction is used as carbon source, wherein side-chain hemicellulolytic enzymes are produced, thereby accelerating subsequent enzymatic hydrolysis and fermentation.

In yet another embodiment, a soluble hemicellulose fraction may be treated with steam, resulting in pretreated substrates that are rich in xylose oligomers, which may be used as inducers for the biosyntheses of hemicellulases.

### BRIEF DESCRIPTION OF THE FIGURES

FIG. 1 depicts schematically a matrix of processes for producing ethanol or other fermentation products from cellulosic substrates, wherein the processing includes progressive fermentation with yeast and thermophilic bacteria.

FIG. 2 depicts schematically a process to produce biofuels or chemicals by progressive fermentation with fungi and bacteria or yeast.

FIG. 3 depicts schematically a process to produce enzymes and ethanol by progressive fermentation with fungi and yeast or bacteria.

FIG. 4 depicts the composition of MTC medium.

FIG. 5 depicts ethanol production in (a) progressive fermentation (squares) and (b) control bacterial fermentation (triangles) of unwashed PHWS (final concentration: 10% TS (w/w)).

FIG. 6 depicts glucose accumulation in (a) progressive fermentation (squares) and (b) control bacterial fermentation (triangles) of unwashed PHWS (final concentration: 10% TS (w/w)).

FIG. 7 depicts T. reesei Rut C30 grown on unwashed pretreated hardwood substrate (MS029, 6% TS (w/w)).

FIG. 8 depicts a comparison of the glucose and cellobiose yields for enzymatic hydrolysis with (a) commercial enzyme (Genencor, Accelerase1000) and (b) the enzymes produced in the T. reesei Rut C30 fermentation (EM2, after 5 days, pretreated hardwood substrate).

FIG. 9 depicts adapted T. reesei Rut C30 grown on unwashed pretreated hardwood substrate (MS149, 15% TS (w/w)).

### DETAILED DESCRIPTION OF THE INVENTION

Aspects of the present invention relate to a process by which the cost of ethanol production from cellulosic biomass-containing materials can be reduced by using a novel processing configuration. It will be appreciated that the present invention utilizes the inherent properties of yeast, fungi, and thermophilic bacteria to reduce the cost of production of cellulosic ethanol.

In one embodiment of the invention, yeast or fungi are added to a reactor containing cellulosic biomass, and the yeast or fungi begins fermentation, thereby completely or partially avoiding the need for oxygen and the production of downstream inhibitors. In one aspect of the invention, the absence of oxygen and inhibitors benefits the subsequent fermentation with a thermophilic bacterium. In yet another embodiment, waste yeast or fungi from the initial stage of the process may be used as a complementary nutrient to enhance the growth of the bacteria. More particularly, the yeast or fungal biomass may be utilized as a nitrogen source to enhance the subsequent fermentation by the thermophilic bacteria.

The terms “progressive fermenting,” “progressive fermentation,” “fermenting,” and “fermentation” are intended to include the enzymatic process (e.g., cellular or acellular (e.g., a lysate or purified polypeptide mixture)) by which ethanol is produced from a carbohydrate, in particular, as a primary product of fermentation.

“Waste material(s)” or “cellulosic waste material(s)” is intended to include any substance comprising cellulose, hemicellulose, or cellulose and hemicellulose. Suitable cellulosic waste materials include, but are not limited to, e.g., corn stover, corn fiber, rice fiber, wheat straw, oat hulls, brewers spent grains, pulp and paper mill waste, wood chips, sawdust, forestry waste, agricultural waste, bagasse, and barley straw.

By “thermophilic” is meant an organism that thrives at a temperature of about 45° C. or higher.

### Biomass

As used herein, the term “biomass” refers to a cellulose-, hemicellulose-, or lignocellulose-containing material. Biomass is commonly obtained from, for example, wood, plants, residue from agriculture or forestry, organic component of municipal and industrial wastes, primary sludges from paper manufacture, waste paper, waste wood (e.g., sawdust), agricultural residues such as corn husks, corn cobs, rice hulls, straw, bagasse, starch from corn, wheat oats, and barley, waste plant material from hard wood or beech bark, fiberboard industry waste water, bagasse pity, bagasse, molasses, post-fermentation liquor, furfural still residues, aqueous oak wood extracts, rice hull, oats residues, wood sugar slops, fir sawdust, naphtha, corncob furfural residue, cotton balls, rice, straw, soybean skin, soybean oil residue, corn husks, cotton stems, cottonseed hulls, starch, potatoes, sweet potatoes, lactose, waste wood pulping residues, sunflower seed husks, hexose sugars, pentose sugars, sucrose from sugar cane and sugar beets, corn syrup, hemp, and combinations of the above.

The terms “lignocellulosic material,” “lignocellulosic substrate,” and “cellulosic biomass” mean any type of biomass comprising cellulose, hemicellulose, lignin, or combinations thereof, such as but not limited to woody biomass, forage grasses, herbaceous energy crops, non-woody-plant biomass, agricultural wastes and/or agricultural residues, forestry residues and/or forestry wastes, paper-production sludge and/or waste paper sludge, waste-water-treatment sludge, municipal solid waste, corn fiber from wet and dry mill corn ethanol plants, and sugar-processing residues.

In a non-limiting example, the lignocellulosic material can include, but is not limited to, woody biomass, such as recycled wood pulp fiber, sawdust, hardwood, softwood, and combinations thereof; grasses, such as switch grass, cord grass, rye grass, reed canary grass, miscanthus, or a combination thereof; sugar-processing residues, such as but not limited to sugar cane bagasse; agricultural wastes, such as but not limited to rice straw, rice hulls, barley straw, corn cobs, cereal straw, wheat straw, canola straw, oat straw, oat hulls, and corn fiber; stover, such as but not limited to soybean stover, corn stover; and forestry wastes, such as but not limited to recycled wood pulp fiber, sawdust, hardwood (e.g., poplar, oak, maple, birch), softwood, or any combination thereof. Lignocellulosic material may comprise one species of fiber; alternatively, lignocellulosic material may comprise a mixture of fibers that originate from different lignocellulosic materials. Particularly advantageous lignocellulosic materials are agricultural wastes, such as cereal straws, including wheat straw, barley straw, canola straw and oat straw; corn fiber; stovers, such as corn stover and soybean stover; grasses, such as switch grass, reed canary grass, cord grass, and miscanthus; or combinations thereof.

Paper sludge is also a viable feedstock for ethanol production. Paper sludge is solid residue arising from pulping and paper-making, and is typically removed from process wastewater in a primary clarifier. At a disposal cost of $30/wet ton, the cost of sludge disposal equates to $5/ton of paper that is produced for sale. The cost of disposing of wet sludge is a significant incentive to convert the material for other uses, such as conversion to ethanol. Processes provided by the present invention are widely applicable. Moreover, the saccharification and/or fermentation products may be used to produce ethanol or higher value added chemicals, such as organic acids, aromatics, esters, acetone and polymer intermediates.

Lignocellulosic materials are composed of mainly cellulose, hemicellulose, and lignin. Generally, a lignocellulosic material, on a dry basis, may contain about 50% (w/w) cellulose, about 30% (w/w) hemicellulose, and about 20% (w/w) lignin. The lignocellulosic material can be of lower cellulose content, for example, at least about 20% (w/w), 30% (w/w), 35% (w/w), or 40% (w/w).

### Reaction Vessel

The term “reactor” may mean any vessel suitable for practicing a method of the present invention. The dimensions of the pretreatment reactor may be sufficient to accommodate the lignocellulose material conveyed into and out of the reactor, as well as additional headspace around the material. In a non-limiting example, the headspace may extend about one foot around the space occupied by the materials. Furthermore, the reactor may be constructed of a material capable of withstanding the pretreatment conditions. Specifically, the construction of the reactor should be such that the pH, temperature and pressure do not affect the integrity of the vessel.

The size range of the substrate material varies widely and depends upon the type of substrate material used as well as the requirements and needs of a given process. In a preferred embodiment of the invention, the lignocellulosic raw material may be prepared in such a way as to permit ease of handling in conveyors, hoppers and the like. In the case of wood, the chips obtained from commercial chippers may be suitable; in the case of straw it may be desirable to chop the stalks into uniform pieces about 1 to about 3 inches in length. Depending on the intended degree of pretreatment, the size of the substrate particles prior to pretreatment may range from less than a millimeter to inches in length. The particles need only be of a size that is reactive.

### Reaction Time

Heating of the lignocellulosic material(s) in the liquid, aqueous medium in the manner according to the invention will normally be carried out for a period of time ranging from about 1 minute to about 1 hour (i.e., about 1-60 minutes), depending not only on the other reaction conditions (e.g., the reaction temperature, and the type and concentration of medium) employed, but also on the reactivity (rate of reaction) of the lignocellulosic material. In certain embodiments of the invention, step (ii) may employ reaction times in the range of 5-30 minutes, often 5-15 minutes, and other reaction conditions, such as an oxygen (partial) pressure may be in the range of about 3-12 bar, e.g., 3-10 bar, and a temperature in the range of about 160-210° C., suitable reaction times will often be in the range of about 10 to about 15 minutes.

### Adjustment of pH in the Reaction Mixture

For some types of lignocellulosic materials of relevance in the context of the invention it may be advantageous to adjust the pH of the reaction mixture before and/or during performance of the treatment. The pH may be decreased, i.e., acidic conditions, but in general the pH of the reaction mixture is increased (i.e., alkaline) by adding appropriate amounts of an alkali or base (e.g., an alkali metal hydroxide such as sodium or potassium hydroxide, an alkaline earth metal hydroxide such as calcium hydroxide, an alkali metal carbonate such as sodium or potassium carbonate or another base such as ammonia) and/or a buffer system. Thus, in certain embodiments of the present invention the aqueous slurry is subjected to alkaline conditions.

In certain embodiment, adjustment of pH may be necessary for one or more steps, and each step may require a different pH or pH range. Accordingly, in one embodiment, for the first fermentation with yeast or fungi, pH may be adjusted to ˜5, while pH may be increased to 6-7 in the second fermentation with bacteria. In certain embodiments, relatively high pH (˜6) is helpful for rapid autolysis of yeast or fungi cells.

### Microorganisms

Thermophilic bacteria or other organisms may be employed in the present invention for the subsequent fermentation to convert all sugars from both hemicellulose and cellulose to ethanol. Thus, aspects of the present invention relate to the use of thermophilic microorganisms. Their potential in process applications in biotechnology stems from their ability to grow at relatively high temperatures with attendant high metabolic rates, production of physically and chemically stable enzymes, and elevated yields of end products. Major groups of thermophilic bacteria include eubacteria and archaebacteria. Thermophilic eubacteria include: phototropic bacteria, such as cyanobacteria, purple bacteria, and green bacteria; Gram-positive bacteria, such as Bacillus, Clostridium, Lactic acid bacteria, and Actinomyces; and other eubacteria, such as Thiobacillus, Spirochete, Desulfotomaculum, Gram-negative aerobes, Gram-negative anaerobes, and Thermotoga. Within archaebacteria are considered Methanogens, extreme thermophiles (an art-recognized term), and Thermoplasma. In certain embodiments, the present invention relates to Gram-negative organotrophic thermophiles of the genera Thermus, Gram-positive eubacteria, such as genera Clostridium, and also which comprise both rods and cocci, genera in group of eubacteria, such as Thermosipho and Thermotoga, genera of Archaebacteria, such as Thermococcus, Thermoproteus (rod-shaped), Thermofilum (rod-shaped), Pyrodictium, Acidianus, Sulfolobus, Pyrobaculum, Pyrococcus, Thermodiscus, Staphylothermus, Desulfurococcus, Archaeoglobus, and Methanopyrus. Some examples of thermophilic microorganisms (including bacteria, procaryotic microorganism, and fungi), which may be suitable for the present invention include, but are not limited to: Clostridium thermosulfurogenes, Clostridium cellulolyticum, Clostridium thermocellum, Clostridium thermohydrosulfuricum, Clostridium thermoaceticum, Clostridium thermosaccharolyticum, Clostridium tartarivorum, Clostridium thermocellulaseum, Thermoanaerobacterium thermosaccarolyticum, Thermoanaerobacterium saccharolyticum, Thermobacteroides acetoethylicus, Thermoanaerobium brockii, Methanobacterium thermoautotrophicum, Pyrodictium occultum, Thermoproteus neutrophilus, Thermofilum librum, Thermothrix thioparus, Desulfovibrio thermophilus, Thermoplasma acidophilum, Hydrogenomonas thermophilus, Thermomicrobium roseum, Thermus flavas, Thermus rubes; Pyrococcus furiosus, Thermus aquaticus, Thermus thermophilus, Chloroflexus aurantiacus, Thermococcus litoralis, Pyrodictium abyssi, Bacillus stearothermophilus, Cyanidium caldarium, Mastigocladus laminosus, Chlamydothrix calidissima, Chlamydothrix penicillata, Thiothrix carnea, Phormidium tenuissimum, Phormidium geysericola, Phormidium subterraneum, Phormidium bijahensi, Oscillatoria filiformis, Synechococcus lividus, Chloroflexus aurantiacus, Pyrodictium brockii, Thiobacillus thiooxidans, Sulfolobus acidocaldarius, Thiobacillus thermophilica, Bacillus stearothermophilus, Cercosulcifer hamathensis, Vahlkampfia reichi, Cyclidium citrullus, Dactylaria gallopava, Synechococcus lividus, Synechococcus elongatus, Synechococcus minervae, Synechocystis aquatilus, Aphanocapsa thermalis, Oscillatoria terebriformis, Oscillatoria amphibia, Oscillatoria germinate, Oscillatoria okenii, Phormidium laminosum, Phormidium parparasiens, Symploca thermalis, Bacillus acidocaldarias, Bacillus coagulans, Bacillus thermocatenalatus, Bacillus licheniformis, Bacillus pamilas, Bacillus macerans, Bacillus circulans, Bacillus laterosporus, Bacillus brevis, Bacillus subtilis, Bacillus sphaericus, Desulfotomaculum nigrificans, Streptococcus thermophilus, Lactobacillus thermophilus, Lactobacillus bulgaricus, Bifidobacterium thermophilum, Streptomyces fragmentosporus, Streptomyces thermonitrificans, Streptomyces thermovulgaris, Pseudonocardia thermophile, Thermoactinomyces vulgaris, Thermoactinomyces sacchari, Thermoactinomyces candidas, Thermomonospora curvata, Thermomonospora viridis, Thermomonospora citrina, Microbispora thermodiastatica, Microbispora aerata, Microbispora bispora, Actinobifida dichotomica, Actinobifida chromogens, Micropolyspora caesia, Micropolyspora faeni, Micropolyspora cectivugida, Micropolyspora cabrobrunea, Micropolyspora thermovirida, Micropolyspora viridinigra, Methanobacterium thermoautothropicum, variants thereof, and/or progeny thereof.

In certain embodiments, the present invention relates to thermophilic bacteria of the genera Thermoanaerobacterium or Thermoanaerobacter, including, but not limited to, species selected from the group consisting of: Thermoanaerobacterium thermosulfurigenes, Thermoanaerobacterium aotearoense, Thermoanaerobacterium polysaccharolyticum, Thermoanaerobacterium zeae, Thermoanaerobacterium xylanolyticum, Thermoanaerobacterium saccharolyticum, Thermoanaerobium brockii, Thermoanaerobacterium thermosaccharolyticum, Thermoanaerobacter thermohydrosulfuricus, Thermoanaerobacter ethanolicus, Thermoanaerobacter brockii, variants thereof, and progeny thereof.

In certain embodiments, the present invention relates to microorganisms of the genera Geobacillus, Saccharococcus, Paenibacillus, Bacillus, and Anoxybacillus, including, but not limited to, species selected from the group consisting of: Geobacillus thermoglucosidasius, Geobacillus stearothermophilus, Saccharococcus caldoxylosilyticus, Saccharoccus thermophilus, Paenibacillus campinasensis, Bacillus flavothermus, Anoxybacillus kamchatkensis, Anoxybacillus gonensis, variants thereof, and progeny thereof.

In one embodiment, the present invention features use of cellulolytic microorganisms in the methods described herein. Several microorganisms determined from literature to be cellulolytic have been characterized by their ability to grow on microcrystalline cellulose as well as a variety of sugars. In a non-limiting example, cellulolytic microorganisms may include Clostridium thermocellum, Clostridium cellulolyticum, Thermoanaerobacterium saccharolyticum, C. stercorarium, C. stercorarium II, Caldiscellulosiruptor kristjanssonii, and C. phytofermentans, variants thereof, and progeny thereof.

Several microorganisms determined from literature to be both cellulolytic and xylanolytic have been characterized by their ability to grow on microcrystalline cellulose and birchwood xylan as well as a variety of sugars. Cellulolytic and xylanolytic microorganism may be used in the present invention, including, but not limited to, Clostridium cellulolyticum, Clostridium stercorarium subs. leptospartum, Caldicellulosiruptor kristjanssonii and Clostridium phytofermentans, variants thereof, and progeny thereof.

In certain embodiments, microbes used in ethanol fermentation, such as yeast, fungi, and Zymomonas mobilis, may also be used in the methods of the invention.

The liquid portion of the output containing residual monomers can be subjected to hydrolysate fermentation to produce ethanol or other fermentation products. For example, yeast or Zymomonas mobilis may be used during the fermentation process.

It will be appreciated that various eukaryotic microorganisms that are classified in the kingdom Fungi may be used in the methods of the present invention. In some embodiments of the invention, the fungi are selected from one or more of the following divisions: Chytridiomycota, Blastocladiomycota, Neocallimastigomycota, Zygomycota, Glomeromycota, Ascomycota, or Basidiomycota. In certain embodiments, genetically modified yeasts or fungi may also be used by the methods described herein. In another embodiment, yeasts or fungi used in the methods of the invention may be resistant to organic acids (e.g., acetic acid), furans (furfural and HMF), lignin degradation products, and other toxins (phenolics, tannin) from biomass and biomass pretreatment. In other embodiments, the invention includes yeasts that are classified in the order Saccharomycetales and yeasts of the divisions Ascomycota and Basidiomycota.

It is further an object of the invention that yeast and fungi may be combined in the methods of the invention.

### Cellulolytic Enzymes

In the methods of the present invention, the cellulolytic enzyme may be any enzyme involved in the degradation of lignocellulose to glucose, xylose, mannose, galactose, and arabinose. The cellulolytic enzyme may be a multicomponent enzyme preparation, e.g., cellulase, a monocomponent enzyme preparation, e.g., endoglucanase, cellobiohydrolase, glucohydrolase, beta-glucosidase, or a combination of multicomponent and monocomponent enzymes. The cellulolytic enzymes may have activity, i.e., hydrolyze cellulose, either in the acid, neutral, or alkaline pH-range.

The cellulolytic enzyme may be of fungal or bacterial origin, which may be obtainable or isolated and purified from microorganisms which are known to be capable of producing cellulolytic enzymes, e.g., species of Humicola, Coprinus, Thielavia, Fusarium, Myceliophthora, Acremonium, Cephalosporium, Scytalidium, Penicillium or Aspergillus (see, for example, EP 458162).

The cellulolytic enzymes used in the methods of the present invention may be produced by fermentation of the above-noted microbial strains on a nutrient medium containing suitable carbon and nitrogen sources and inorganic salts, using procedures known in the art (see, e.g., Bennett, J. W. and LaSure, L. (eds.), More Gene Manipulations in Fungi, Academic Press, CA, 1991). Suitable media are available from commercial suppliers or may be prepared according to published compositions (e.g., in catalogues of the American Type Culture Collection). Temperature ranges and other conditions suitable for growth and cellulase production are known in the art (see, e.g., Bailey, J. E., and Ollis, D. F., Biochemical Engineering Fundamentals, McGraw-Hill Book Company, NY, 1986).

### Additional Enzymes

In the methods of the present invention, the cellulolytic enzyme(s) may be supplemented by one or more additional enzyme activities to improve the degradation of the lignocellulosic material. Such additional enzymes may include, for example, hemicellulases, lignin degradation enzymes, esterases (e.g., lipases, phospholipases, and/or cutinases), proteases, laccases, peroxidases, or mixtures thereof.

In the methods of the present invention, the additional enzyme(s) may be added prior to or during fermentation, including during or after the propagation of the fermenting microorganism(s).

The enzymes referenced herein may be derived or obtained from any suitable origin, including, bacterial, fungal, yeast or mammalian origin. As used herein, the term “obtained” means that the enzyme may have been isolated from an organism which naturally produces the enzyme as a native enzyme. The enzymes referenced herein may also refer to the whole broth from enzyme production, including free enzymes, cellular enzymes, and organism cells that produce enzymes. The term “obtained” also means that the enzyme may have been produced recombinantly in a host organism, wherein the recombinantly produced enzyme is either native or foreign to the host organism or has a modified amino acid sequence, e.g., having one or more amino acids which are deleted, inserted and/or substituted, i.e., a recombinantly produced enzyme which is a mutant and/or a fragment of a native amino acid sequence or an enzyme produced by nucleic acid shuffling processes known in the art. Encompassed within the meaning of a native enzyme are natural variants and within the meaning of a foreign enzyme are variants obtained recombinantly, such as by site-directed mutagenesis or shuffling.

The enzymes may also be purified. The term “purified” as used herein covers enzymes free from other components from the organism from which it is derived. The term “purified” also covers enzymes free from components from the native organism from which it is obtained. The enzymes may be purified, with only minor amounts of other proteins being present. The expression “other proteins” relate in particular to other enzymes. The term “purified” as used herein also refers to removal of other components, particularly other proteins and most particularly other enzymes present in the cell of origin of the enzyme of the invention. The enzyme may be “substantially pure,” that is, free from other components from the organism in which it is produced, that is, for example, a host organism for recombinantly produced enzymes. In preferred embodiment, the enzymes are at least 75% (w/w), preferably at least 80%, more preferably at least 85%, more preferably at least 90%, more preferably at least 95%, more preferably at least 96%, more preferably at least 97%, even more preferably at least 98%, or most preferably at least 99% pure. In another preferred embodiment, the enzyme is 100% pure.

The enzymes used in the present invention may be in any form suitable for use in the processes described herein, such as, for example, in the form of a dry powder or granulate, a non-dusting granulate, a liquid, a stabilized liquid, or a protected enzyme. Granulates may be produced, e.g., as disclosed in U.S. Pat. Nos. 4,106,991 and 4,661,452, and may optionally be coated by process known in the art. Liquid enzyme preparations may, for instance, be stabilized by adding stabilizers such as a sugar, a sugar alcohol or another polyol, and/or lactic acid or another organic acid according to established process.

### Hemicellulases

Enzymatic hydrolysis of hemicelluloses can be performed by a wide variety of fungi and bacteria. Similar to cellulose degradation, hemicellulose hydrolysis requires coordinated action of many enzymes. Hemicellulases can be placed into three general categories: the endo-acting enzymes that attack internal bonds within the polysaccharide chain, the exo-acting enzymes that act processively from either the reducing or nonreducing end of polysaccharide chain, and the accessory enzymes, such as acetylesterases and esterases that hydrolyze lignin glycoside bonds, such as coumaric acid esterase and ferulic acid esterase (Wong, K. K. Y., Tan, L. U. L., and Saddler, J. N., 1988, Multiplicity of β-1,4-xylanase in microorganisms: Functions and applications, Microbiol. Rev. 52: 305-317; Tenkanen, M., and Poutanen, K., 1992, Significance of esterases in the degradation of xylans, in Xylans and Xylanases, Visser, J., Beldman, G., Kuster-van Someren, M. A., and Voragen, A. G. J., eds., Elsevier, New York, N.Y., 203-212; Coughlan, M. P., and Hazlewood, G. P., 1993, Hemicellulose and hemicellulases, Portland, London, UK; Brigham, J. S., Adney, W. S., and Himmel, M. E., 1996, Hemicellulases: Diversity and applications, in Handbook on Bioethanol: Production and Utilization, Wyman, C. E., ed., Taylor & Francis, Washington, D.C., 119-141).

Hemicellulases include xylanases, arabinofuranosidases, acetyl xylan esterase, glucuronidases, endo-galactanase, mannanases, endo or exo arabinases, exo-galactanses, and mixtures thereof. Examples of endo-acting hemicellulases and ancillary enzymes include endoarabinanase, endoarabinogalactanase, endoglucanase, endomannanase, endoxylanase, and feraxan endoxylanase. Examples of exo-acting hemicellulases and ancillary enzymes include α-L-arabinosidase, β-L-arabinosidase, α-1,2-L-fucosidase, α-D-galactosidase, β-D-galactosidase, β-D-glucosidase, β-D-glucuronidase, β-D-mannosidase, β-D-xylosidase, exoglucosidase, exocellobiohydrolase, exomannobiohydrolase, exomannanase, exoxylanase, xylan .alpha.-glucuronidase, and coniferin .beta.-glucosidase. Examples of esterases include acetyl esterases (acetylgalactan esterase, acetylmannan esterase, and acetylxylan esterase) and aryl esterases (coumaric acid esterase and ferulic acid esterase).

Preferably, the hemicellulase is an exo-acting hemicellulase, and more preferably, an exo-acting hemicellulase which has the ability to hydrolyze hemicellulose under acidic conditions of below pH 7. An example of a hemicellulase suitable for use in the present invention includes VISCOZYME™ (available from Novozymes A/S, Denmark). The hemicellulase may be added in an effective amount from about 0.001% to about 5.0% wt. of solids, in other embodiments, from about 0.025% to about 4.0% wt. of solids, and still other embodiments, from about 0.005% to about 2.0% wt. of solids.

A xylanase (E.C. 3.2.1.8) may be obtained from any suitable source, including fungal and bacterial organisms, such as Aspergillus, Disporotrichum, Penicillium, Neurospora, Fusarium, Trichoderma, Humicola, Thermomyces, and Bacillus.

### Processing of Lignocellulosic Materials

The methods of the present invention may be used to process a lignocellulosic material to many useful organic products, chemicals and fuels. In addition to ethanol, some commodity and specialty chemicals that can be produced from lignocellulose include xylose, acetone, acetate, glycine, lysine, organic acids (e.g., lactic acid), 1,3-propanediol, butanediol, glycerol, ethylene glycol, furfural, polyhydroxyalkanoates, cis, cis-muconic acid, and animal feed (Lynd, L. R., Wyman, C. E., and Gerngross, T. U., 1999, Biocommodity engineering, Biotechnol. Prog., 15: 777-793; Philippidis, G. P., 1996, Cellulose bioconversion technology, in Handbook on Bioethanol: Production and Utilization, Wyman, C. E., ed., Taylor & Francis, Washington, D.C., 179-212; and Ryu, D. D. Y., and Mandels, M., 1980, Cellulases: biosynthesis and applications, Enz. Microb. Technol., 2: 91-102). Potential coproduction benefits extend beyond the synthesis of multiple organic products from fermentable carbohydrate. Lignin-rich residues remaining after biological processing can be converted to lignin-derived chemicals, or used for power production.

Conventional methods used to process the lignocellulosic material in accordance with the methods of the present invention are well understood to those skilled in the art. The methods of the present invention may be implemented using any conventional biomass processing apparatus configured to operate in accordance with the invention.

Such an apparatus may include a batch-stirred reactor, a continuous flow stirred reactor with ultrafiltration, a continuous plug-flow column reactor (Gusakov, A. V., and Sinitsyn, A. P., 1985, Kinetics of the enzymatic hydrolysis of cellulose: 1. A mathematical model for a batch reactor process, Enz. Microb. Technol., 7: 346-352), an attrition reactor (Ryu, S. K., and Lee, J. M., 1983, Bioconversion of waste cellulose by using an attrition bioreactor, Biotechnol. Bioeng., 25: 53-65), or a reactor with intensive stirring induced by an electromagnetic field (Gusakov, A. V., Sinitsyn, A. P., Davydkin, I. Y., Davydkin, V. Y., Protas, O. V., 1996, Enhancement of enzymatic cellulose hydrolysis using a novel type of bioreactor with intensive stirring induced by electromagnetic field, Appl. Biochem. Biotechnol., 56: 141-153).

The conventional methods include, but are not limited to, saccharification, fermentation, separate hydrolysis and fermentation (SHF), simultaneous saccharification and fermentation (SSF), simultaneous saccharification and cofermentation (SSCF), hybrid hydrolysis and fermentation (HHF), and direct microbial conversion (DMC).

SHF uses separate process steps to first enzymatically hydrolyze cellulose to glucose and then ferment glucose to ethanol. In SSF, the enzymatic hydrolysis of cellulose and the fermentation of glucose to ethanol is combined in one step (Philippidis, G. P., 1996, Cellulose bioconversion technology, in Handbook on Bioethanol: Production and Utilization, Wyman, C. E., ed., Taylor & Francis, Washington, D.C., 179-212). SSCF includes the cofermentation of multiple sugars (Sheehan, J., and Himmel, M., 1999, Enzymes, energy and the environment: A strategic perspective on the U.S. Department of Energy's research and development activities for bioethanol, Biotechnol. Prog., 15: 817-827). HHF includes two separate steps carried out in the same reactor but at different temperatures, i.e., high temperature enzymatic saccharification followed by SSF at a lower temperature that the fermentation strain can tolerate. DMC combines all three processes (cellulase production, cellulose hydrolysis, and fermentation) in one step (Lynd, L. R., Weimer, P. J., van Zyl, W. H., and Pretorius, I. S., 2002, Microbial cellulose utilization: Fundamentals and biotechnology, Microbiol. Mol. Biol. Reviews, 66: 506-577).

“Fermentation” or “fermentation process” refers to any fermentation process or any process comprising a fermentation step. A fermentation process includes, without limitation, fermentation processes used to produce fermentation products including alcohols (e.g., arabinitol, butanol, ethanol, glycerol, methanol, 1,3-propanediol, sorbitol, and xylitol); organic acids (e.g., acetic acid, acetonic acid, adipic acid, ascorbic acid, citric acid, 2,5-diketo-D-gluconic acid, formic acid, fumaric acid, glucaric acid, gluconic acid, glucuronic acid, glutaric acid, 3-hydroxypropionic acid, itaconic acid, lactic acid, malic acid, malonic acid, oxalic acid, propionic acid, succinic acid, and xylonic acid); ketones (e.g., acetone); amino acids (e.g., aspartic acid, glutamic acid, glycine, lysine, serine, and threonine); gases (e.g., methane, hydrogen (H₂), carbon dioxide (CO₂), and carbon monoxide (CO)). Fermentation processes also include fermentation processes used in the consumable alcohol industry (e.g., beer and wine), dairy industry (e.g., fermented dairy products), leather industry, and tobacco industry.

### Enzymatic Hydrolysis of Cellulose and Fermentation of Glucose: Simultaneous Saccharification and Fermentation Process (SSF)

Enzymatic hydrolysis of cellulose is carried out by means of a mixture of enzymatic activities that are known as a group as cellulolytic enzymes or cellulases. One of the enzymes, called endoglucanase, is absorbed on the surface of the cellulose and attacks the inside of the polymer chain, breaking it at one point. A second enzyme, called exoglucanase, subsequently frees two units of glucose, called cellobiose, from the non-reducing end of the chain. The cellobiose produced in this reaction can accumulate in the medium and significantly inhibit the exoglucanase activity. The third enzymatic activity, the β-glucosidase, splits these two sugar units to free the glucose that is later fermented to ethanol. Once again, the glucose can accumulate in the medium and inhibit the effect of the β-glucosidase, then producing an accumulation of cellobiose, which inhibits the exoglucanase activity.

Although there are different types of micro-organisms that can produce cellulases, including bacteria and different kinds of fungi, genetically altered strains of the filamentous fungus Trichoderma ressei may be used, since they have greater yields. Traditional cellulase production methods are discontinuous, using insoluble sources of carbon, both as inducers and as substrates, for the growth of the fungus and enzyme production. In these systems, the speed of growth and the rate of cellulase production are limited, because the fungus has to secrete the cellulases and carry out a slow enzymatic hydrolysis of the solid to obtain the necessary carbon. The best results have generally been obtained in operations with discontinuous feeding, in which the solid substrate, for example Solka Floc or pre-treated biomass, is slowly added to the fermentation deposit so that it does not contain too much substrate (Watson et al., Biotech. Lett., 6, 667, 1984). According to Wright, J. D. (SERI/TP-231-3310, 1988), average productivity using Solka Floc and pre-treated agricultural residues is around 50 IU/l.h.

In the conventional method for producing ethanol from lignocellulosic materials, a cellulase is added to the material pre-treated in a reactor for the saccharification of the cellulose to glucose, and once this reaction is completed, the glucose is fermented to ethanol in a second reactor. This process, called separate saccharification and fermentation, implies two different stages in the process of obtaining ethanol. Using this method, the conversion rate of cellulose to glucose is low, because of the inhibition that the accumulation of glucose and cellobiose causes to the action of the enzyme complex, and consequently, large amounts of non-hydrolysed cellulosic residues are obtained which have a low ethanol yield. This inhibition of the final product is one of the most significant disadvantages of the separate saccharification and fermentation process, and is one of the main factors responsible for its high cost, since large amounts of cellulolytic enzyme are used in an attempt to solve this problem.

Simultaneous saccharification and fermentation (SSF) is a process by which the presence of yeast, bacteria, or other organisms, together with the cellulolytic enzyme, reduces the accumulation of sugars in the reactor and it is therefore possible to obtain greater yields and saccharification rates than with the separate hydrolysis and fermentation process. Another advantage is the use of a single fermentation deposit for the entire process, thus reducing the cost of the investment involved. The presence of ethanol in the medium may also makes the mixture less liable to be invaded by undesired microorganisms.

In the simultaneous hydrolysis and fermentation process, the fermentation and saccharification must be compatible and have a similar pH, temperature and optimum substrate temperature. One problem associated to the SSF process is the different optimum temperatures for saccharification and fermentation.

### Methods of the Invention

During glycolysis, cells convert simple sugars, such as glucose, into pyruvic acid, with a net production of ATP and NADH. In the absence of a functioning electron transport system for oxidative phosphorylation, at least 95% of the pyruvic acid is consumed in short pathways which regenerate NAD⁺, an obligate requirement for continued glycolysis and ATP production. The waste products of these NAD⁺ regeneration systems are commonly referred to as fermentation products.

Microorganisms produce a diverse array of fermentation products, including organic acids, such as lactate, acetate, succinate, and butyrate, and neutral products, such as ethanol, butanol, acetone, and butanediol. End products of fermentation share several fundamental features: they are relatively nontoxic under the conditions in which they are initially produced, but become more toxic upon accumulation; and they are more reduced than pyruvate because their immediate precursors have served as terminal electron acceptors during glycolysis.

It is one aspect of the invention that yeast fermentation, yeast autolysis, and bacteria fermentation can be carried out in the same vessel or different vessels. Furthermore, the processes contemplated herein can be in batch, fed-batch/semi-continuous, or continuous operations. Multistage continuous fermentation is highly recommended for its convenience for reaction control, high solid fermentation, and ethanol productivity.

### Exemplary Embodiments

According to one embodiment of the present invention, there is provided a method of processing lignocellulosic material, comprising the steps of: placing a sample of lignocellulosic material in a reactor; adding to said reactor a yeast or fungus at a first temperature and pH to give a first mixture; adding to said first mixture a thermophilic microorganism and at least one enzyme at a second temperature and pH to give a second mixture; and allowing the second mixture to age for a period of time to give a third mixture; wherein said third mixture comprises a liquid product and a solid product; and said liquid product comprises ethanol and other fermentation products.

In certain embodiments, the present invention relates to the aforementioned method, further comprising the step of recovering the ethanol.

In certain embodiments, the present invention relates to the aforementioned method, wherein yeast and fungus are added to said reactor at a first temperature and pH.

In certain embodiments, the present invention relates to the aforementioned method, wherein said at least one enzyme is a cellulolytic enzyme.

In certain embodiments, the present invention relates to the aforementioned method, wherein said cellulolytic enzyme is selected from the group consisting of a cellulase, endoglucanase, cellobiohydrolase, and beta-glucosidase.

In certain embodiments, the present invention relates to the aforementioned method, further comprising treating the lignocellulosic material with an effective amount of at least one enzyme selected from the group consisting of a hemicellulase, esterase, protease, laccase, and peroxidase.

In certain embodiments, the present invention relates to the aforementioned method, wherein said second temperature is above 45° C.

In certain embodiments, the present invention relates to the aforementioned method, wherein said second temperature is above 50° C.

In certain embodiments, the present invention relates to the aforementioned method, wherein said second temperature is about 55° C.

In certain embodiments, the present invention relates to the aforementioned method, wherein the first pH is about 5.

In certain embodiments, the present invention relates to the aforementioned method, wherein the second pH is between 5-6.

In certain embodiments, the present invention relates to the aforementioned method, wherein the second pH is between 6-7.

In certain embodiments, the present invention relates to the aforementioned method, wherein the second pH is greater than 6.

In certain embodiments, the present invention relates to the aforementioned method, wherein said yeast or fungus removes inhibitors in said reactor.

In certain embodiments, the present invention relates to the aforementioned method, wherein said inhibitors comprise acetate, furfural, HMF, phenolics, and lignin degradation products.

In certain embodiments, the present invention relates to the aforementioned method, wherein said yeast or fungi perform fermentation.

In certain embodiments, the present invention relates to the aforementioned method, wherein said yeast or fungi undergo autolysis.

In certain embodiments, the present invention relates to the aforementioned method, wherein said autolysis of the yeast or fungi produces enzymes or proteins.

In certain embodiments, the present invention relates to the aforementioned method, wherein said thermophilic microorganism is a bacterium; and the bacteria perform fermentation.

In certain embodiments, the present invention relates to the aforementioned method, wherein said autolyzed yeast or fungi may be utilized by said microorganism for growth.

In certain embodiments, the present invention relates to the aforementioned method, wherein the enzymes or proteins produced from the autolyzed yeast or fungi may be utilized as supplemental enzymes.

According to one embodiment of the present invention, there is provided a method for converting lignocellulosic biomass material into ethanol, the method comprising the steps of:

(i) preparing in a reaction vessel an aqueous slurry of said biomass material;

(ii) adding to said reaction vessel a yeast or fungus resulting in partial separation of the biomass material into cellulose, hemicellulose and lignin;

(iii) adding to said reaction vessel a thermophilic microorganism and at least one enzyme;

(iv) heating for a period of time said reaction vessel to give a mixture;

wherein said mixture comprises a liquid product and a solid product; and said liquid product comprises ethanol.

In certain embodiments, the present invention relates to the aforementioned method,

wherein the treatment of step (iii) is an anaerobic fermentation process.

In certain embodiments, the present invention relates to the aforementioned method, further comprising pretreating said aqueous slurry in said reaction vessel.

In certain embodiments, the present invention relates to the aforementioned method, wherein the steps are performed as a batch process in a closed, pressurizable reaction vessel having a free volume for containing oxygen-containing gas or water vapor with or without additional gasses.

In certain embodiments, the present invention relates to the aforementioned method, wherein the steps are performed as a batch process in a closed, pressurizable reaction vessel with recirculation of the reaction mixture.

In certain embodiments, the present invention relates to the aforementioned method, wherein the steps are performed as a continuous process in an essentially tubular reactor.

In certain embodiments, the present invention relates to the aforementioned method, wherein step (iii) is performed at an elevated temperature of greater than 50° C.

In certain embodiments, the present invention relates to the aforementioned method, wherein step (iii) is performed at an elevated temperature of about 55° C.

In certain embodiments, the present invention relates to the aforementioned method, wherein step (iii) is performed at an elevated temperature of greater than 100° C.

In certain embodiments, the present invention relates to the aforementioned method, wherein said lignocellulosic material contains, on a dry basis, at least about 20% (w/w) cellulose, at least about 10% (w/w) hemicellulose, and at least about 10% (w/w) lignin.

In certain embodiments, the present invention relates to the aforementioned method, wherein said lignocellulosic material is selected from the group consisting of grass, switch grass, cord grass, rye grass, reed canary grass, miscanthus, sugar-processing residues, sugar cane bagasse, agricultural wastes, rice straw, rice hulls, barley straw, corn cobs, cereal straw, wheat straw, canola straw, oat straw, oat hulls, corn fiber, stover, soybean stover, corn stover, forestry wastes, recycled wood pulp fiber, sawdust, hardwood, and softwood.

In certain embodiments, the present invention relates to the aforementioned method, wherein said lignocellulosic material is hardwood; and said hardwood is selected from the group consisting of willow, maple, oak, walnut, eucalyptus, elm, birch, buckeye, beech, and ash.

In certain embodiments, the present invention relates to the aforementioned method, wherein said lignocellulosic material is hardwood, and said hardwood is willow.

In certain embodiments, the present invention relates to the aforementioned method, wherein said lignocellulosic material is softwood; and said softwood is selected from the group consisting of southern yellow pine, fir, cedar, cypress, hemlock, larch, pine, and spruce.

In certain embodiments, the present invention relates to the aforementioned method, wherein said lignocellulosic material is softwood, and said softwood is southern yellow pine.

In certain embodiments, the present invention relates to the aforementioned method, wherein the yeast is selected from the group consisting of Ascomycota, Basidiomycota or Saccharomycetales.

In certain embodiments, the present invention relates to the aforementioned method, wherein the yeast is highly resistant to inhibitors.

In certain embodiments, the present invention relates to the aforementioned method, wherein the yeast is genetically engineered or naturally capable of metabolizing the inhibitors.

In certain embodiments, the present invention relates to the aforementioned method, wherein the thermophilic microorganism is a species of the genera Thermoanaerobacterium, Thermoanaerobacter, Clostridium, Geobacillus, Saccharococcus, Paenibacillus, Bacillus, or Anoxybacillus.

In certain embodiments, the present invention relates to the aforementioned method, wherein the thermophilic microorganism is a bacterium selected from the group consisting of: Thermoanaerobacterium thermosulfurigenes, Thermoanaerobacterium aotearoense, Thermoanaerobacterium polysaccharolyticum, Thermoanaerobacterium zeae, Thermoanaerobacterium xylanolyticum, Thermoanaerobacterium saccharolyticum, Thermoanaerobium brockii, Thermoanaerobacterium thermosaccharolyticum, Thermoanaerobacter thermohydrosulfuricus, Thermoanaerobacter ethanolicus, Thermoanaerobacter brocki, Clostridium thermocellum, Geobacillus thermoglucosidasius, Geobacillus stearothermophilus, Saccharococcus caldoxylosilyticus, Saccharoccus thermophilus, Paenibacillus campinasensis, Bacillus flavothermus, Anoxybacillus kamchatkensis, and Anoxybacillus gonensis.

In certain embodiments, the present invention relates to the aforementioned method, wherein the fungus is selected from the group consisting of Chytridiomycota, Blastocladiomycota, Neocallimastigomycota, Zygomycota, Glomeromycota, Ascomycota, Basidiomycota, and T. reesei Rut 30.

In certain embodiments, the present invention relates to the aforementioned method, wherein step (ii) comprises adding to said reaction vessel yeast and fungus.

In certain embodiments, the present invention relates to the aforementioned method, further comprising the step of subjecting said liquid product to hydrolysate fermentation.

In certain embodiments, the present invention relates to the aforementioned method, further comprising the step of subjecting said solid product to autohydrolysis pretreatment.

In certain embodiments, the present invention relates to the aforementioned method, wherein the autohydrolysis pretreatment is steam hydrolysis.

In certain embodiments, the present invention relates to the aforementioned method, wherein the autohydrolysis pretreatment is acid hydrolysis.

In certain embodiments, the present invention relates to the aforementioned method, further comprising the step of subjecting said solid product to consolidated bioprocessing.

### EXEMPLIFICATION

### Example 1

### Progressive Fermentation with Yeast and Thermophilic Bacteria

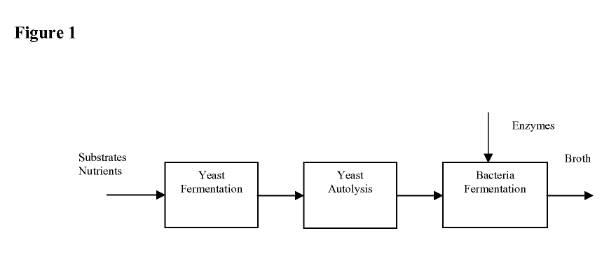

As described herein, the methods of the present invention use progressive fermentation of yeast and thermophilic bacteria to produce ethanol from cellulosic substrates. FIG. 1 depicts schematically a matrix of processes for producing ethanol and other fermentation products from cellulosic substrates, the processing including progressive fermentation of yeast and thermophilic bacteria, according to the methods of the invention. As shown in FIG. 1, the medium containing substrates and nutrients, may be inoculated with yeast to completely or partially remove oxygen and inhibitors that are present in solid substrates or hydrolyzates from biomass pretreatment. At the same time, hemicellulose sugars may be partially fermented into ethanol, when pentose fermenting yeast is used. The temperature and pH of the broth from the first fermentation stage are then adjusted to accelerate the autolysis of yeast. Enzymes, such as cellulases and hemicellulases, supplemental nutrients, and thermophilic bacteria, are added to convert all hemicellulose sugars and cellulose to ethanol.

The substrates used herein can be woody biomass (softwood and hardwood), herbaceous plants (e.g., grasses, herbaceous energy crops, bamboos), agricultural residues (e.g., corn stover, rice straw, wheat stalk), and other fiber wastes (grain fibers, fruits fiber, and municipal wastes).

Yeast used according to the methods of the invention may be resistant to organic acids (e.g., acetic acid), furans (furfural and HMF), lignin degradation products, and other toxins (phenolics, tannin) from biomass and biomass pretreatment. Thermophilic bacteria or other organisms are employed for the subsequent fermentation to convert all sugars from both hemicellulose and cellulose to ethanol.

It is one aspect of the invention that yeast fermentation, yeast autolysis, and bacteria fermentation can be carried out in the same vessel or different vessels. Furthermore, the processes contemplated herein can be in batch, fed-batch/semi-continuous, or continuous operations. Multistage continuous fermentation is a highly recommended for its convenience for reaction control, high solid fermentation, and ethanol productivity.

It will be appreciated that detoxification by yeast using the methods described herein may be further improved by microbiology and molecular biology approaches that are known in the art. In addition, it is an aspect of the invention to use organisms that have a naturally high inhibitory tolerance and are found in nature.

It is also an aspect of the invention to reduce and/or remove byproducts or inhibitors of yeast fermentation or yeast autolysis throughout the methods described herein.

### Example 2

### Progressive Fermentation with Fungi and Thermophilic Bacteria

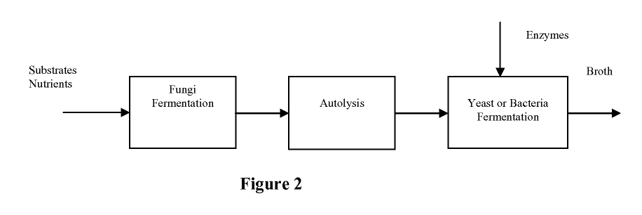

Some fungi such as Trichodema, Penicillium or Aspergillus have a high tolerance to inhibitors such as acetate, furfural, HMF, and phenolics that are commonly present in the pretreated substrates or hydrolyzates, and can metabolize parts of the inhibitors by fermentation. At the same time, most fungi produce hydrolytic enzymes including cellulases and hemicellulases that are required to hydrolyze cellulose and hemicellulose to sugars. FIG. 2 shows the schematic process for biological conversion of cellulosic biomass to biofuels or chemicals. Inhibitors present in the cellulosic substrates will be partially removed by fermentation with fungi, followed by simultaneous saccharification and fermentation with addition of yeast or bacteria, and enzymes to produce target products.

It will be appreciated that detoxification by fungi using the methods described herein may be further improved by microbiology and molecular biology approaches that are known in the art. In addition, it is an aspect of the invention to use organisms that have a naturally high inhibitory tolerance and are found in nature.

It is also an aspect of the invention to reduce and/or remove byproducts or inhibitors of fungi fermentation or fungi autolysis throughout the methods described herein.

### Example 3

### Progressive Fermentation to Produce Enzymes and Ethanol

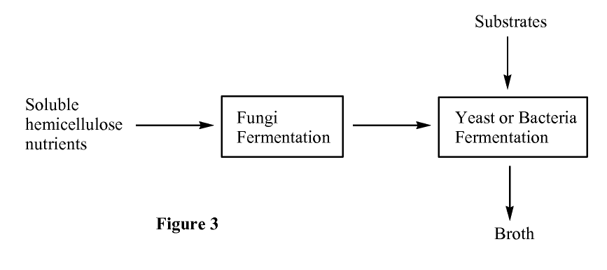

Cellulases and hemicellulases are expensive and required enzymes in the cellulosic ethanol process; however, both enzymes can be produced effectively and inexpensively based on the processes depicted in FIG. 3. By removing the soluble fraction from pretreated substrates with hot water, there would be an increase in cellulose digestibility at reduced enzyme loadings. This process would also enhance SSF of the solids and fermentability of the hydrolyzates for the partial removal of lignin and inhibitors.

In one aspect, the invention features a soluble hemicellulose fraction in pretreated substrates that is separated by hot washing and may be used as a carbon source to produce hemicellulases by fungi, such as T. reesei Rut 30. The whole broth comprising fungi cells and produced enzymes may be used for subsequent enzymatic hydrolysis and fermentation. Accordingly, by using a soluble hemicellulose fraction as carbon source, side-chain hemicellulolytic enzymes will be produced, thereby accelerating subsequent enzymatic hydrolysis and fermentation.

In certain embodiments, a soluble hemicellulose fraction may be treated with steam, resulting in pretreated substrates that are rich in xylose oligomers, which are good inducers for the biosyntheses of hemicellulases. By combining the fungi cells and the produced enzymes to perform enzymatic hydrolysis and fermentation, the enzymes work more efficiently.

### Example 4

### Progressive Fermentation with Yeast and Thermophilic Bacteria

C6-fermenting yeast and Mascoma-engineered thermophilic T. sacch were used to evaluate the performance of the yeast-to-bacteria progressive fermentation process. Unwashed PHWS (MS149) (5 g, dry weight) was loaded in a 250-mL pressure bottle and autoclaved at 121° C. for 30 min. Sterile 5×YP medium (5 mL), glucose solution (5 mL, 10 g/L), and DI water (10 mL) were then added. The system was then inoculated with fresh yeast culture (5 mL, OD 600 nm ˜5), yielding a system with a final concentration (w/w) of 12.5% TS substrate, 1% yeast, 2% tryptone, and 0.1% glucose.

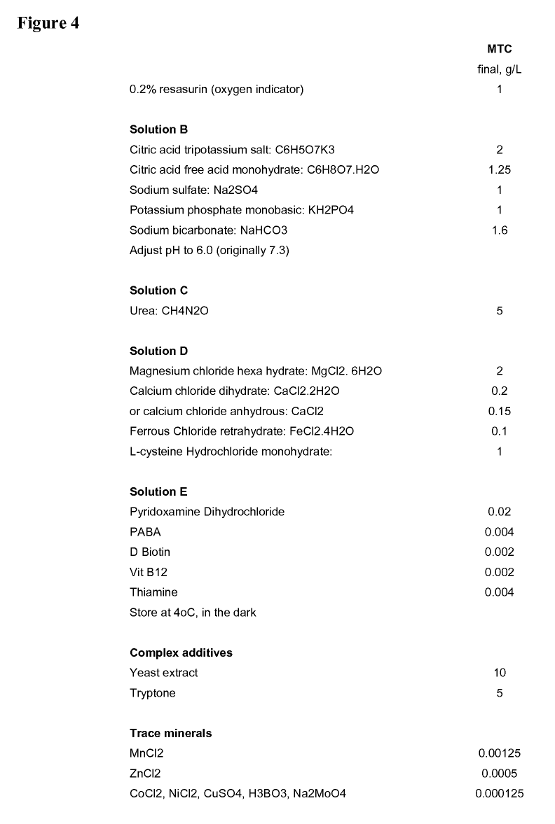

The first fermentation was performed at 30° C. and 200 rpm for 3 days. Subsequently, the system was incubated at elevated temperature (55° C.) for 3-5 hours to lyze the yeast. After the yeast lysis, 5.6×MTC medium (8 mL, FIG. 4) and enzyme (2.5 mL, Mix B, 20 mg total protein per mL) were added. The system was purged with N₂ to remove the oxygen in the bottle. Finally, T. sacch culture (5 mL, OD 600 nm ˜5) was added, with the final substrate concentration decreased to about 10% TS (w/w). The second fermentation was performed at 55° C., pH ˜5.5, and 200 rpm.

A control experiment was run: unwashed PHWS (MS149) (5 g, dry weight) was loaded in a 250-mL pressure bottle and autoclaved at 121° C. for 30 min. Sterile 5×YP medium (5 mL), glucose solution (5 mL, 10 g/L), and DI water (10 mL) were then added. The system was NOT inoculated with fresh yeast culture. All other experimental conditions remained the same.

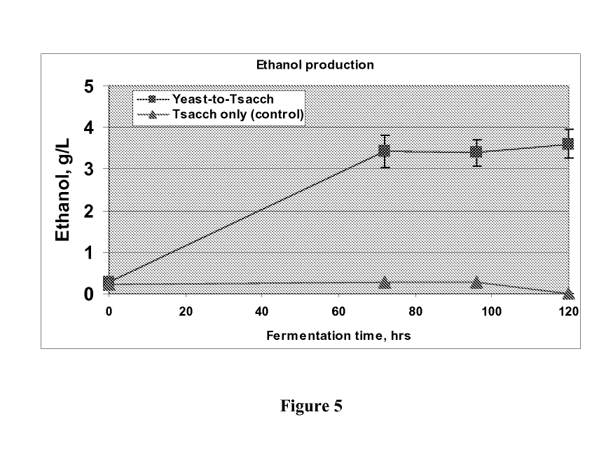

Each experiment was run in duplicate. Ethanol and residual glucose were determined by HPLC. As presented in FIG. 5, no ethanol was produced in the control fermentation, indicating that T. sacch did not grow on the unwashed substrate at this high concentration of solids. Our previous data have shown that the T. sacch test strain can only grow on the unwashed PHWS at a solid concentration less than 5% TS (w/w). However, the experiment showed that, after 3 days of yeast fermentation, the T. sacch test strain was able to ferment the substrate at the same solid concentration (10% TS (w/w)) (FIG. 5). Therefore, yeast fermentation reduced the negative impact of inhibitors (present in the substrate) on T. sacch; the bacteria were more easily able to ferment the substrate after yeast fermentation.

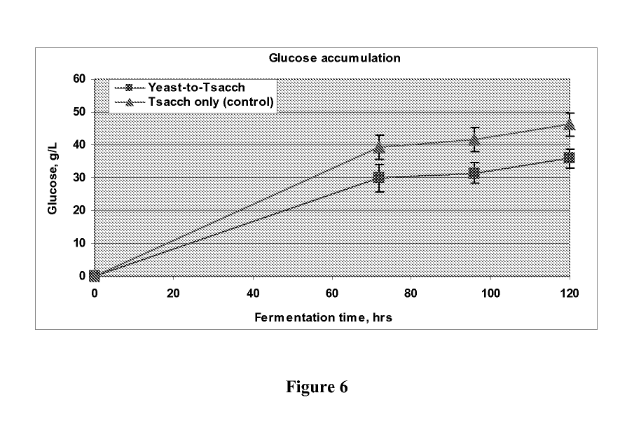

However, the T. sacch fermentation (TSSCF) was still very slow in this experiment. One possible explanation for the low bacterial fermentation rate is that the yeast fermentation was performed in a pressure bottle with limited oxygen. This may have decreased the ability of the yeast to metabolize the inhibitors present in the substrate. Because the bacterial fermentation was very slow, high concentrations of glucose were observed (FIG. 6).

### Example 5

### Progressive Fermentation with Fungi and Yeast or Bacteria

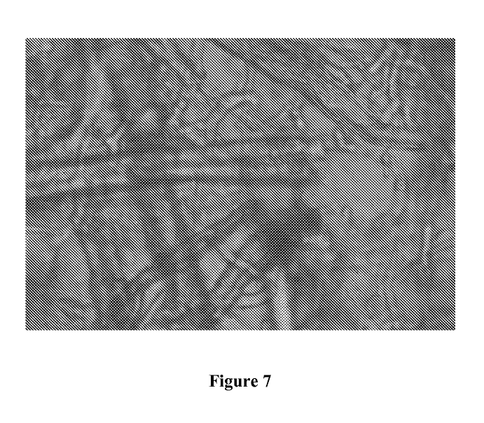

In this experiment, T. reesei Rut C30 from ATT was used as the microorganism in the first fermentation of the progressive fermentation process. Unwashed pretreated hardwood substrate (MS029) was used. The first fermentation mixture also included: 0.07% (NH₄)₂SO₄, 0.15% urea, and 0.5% soybean flour. Batch fermentation was conducted in a shaking flask under the following conditions: 6% TS (w/w), initial pH ˜4.8, 30° C., and 200 rpm. As depicted in FIG. 7, this organism grew very well on this substrate at this solid concentration.

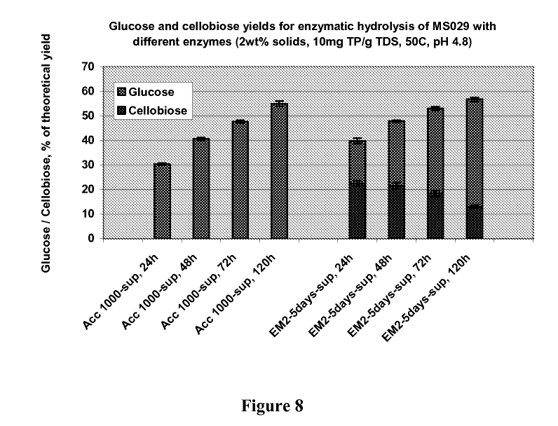

Many enzymes were produced during this fermentation. Surprisingly, these enzymes proved to be more effective for hydrolysis of the substrate than commercial enzymes (FIG. 8). Thus, T. reesei fermentation not only removed some of the inhibitors present in the substrate, but also provided supplemental enzymes for subsequent SSF for ethanol production.

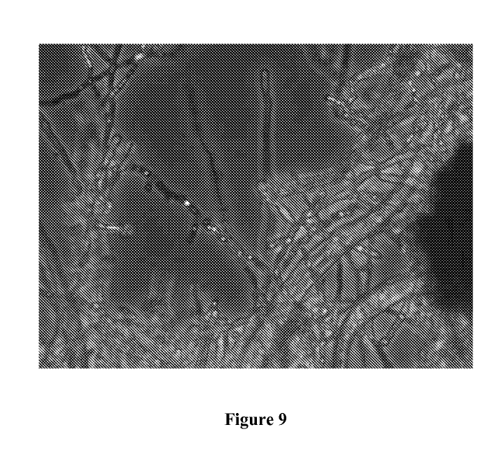

The tolerance of T. reesei to inhibitors was significantly increased by series tube transfer. FIG. 9 presents the adapted strain that grew on unwashed pretreated hardwood substrate at a solid concentration up to 15% TS (w/w).

In the future, the inhibitor tolerances of the microorganisms and their growth rates at high solid concentrations will be increased. The ability of the adapted T. reesei strain to metabolize inhibitors and to produce cellulolytic enzymes will be examined. Additionally, the performance of the T. reesei-to-T. sacch progressive fermentation process for ethanol production will be explored.

### EQUIVALENTS

Those skilled in the art will recognize, or be able to ascertain using no more than routine experimentation, many equivalents to the specific embodiments of the invention described herein. Such equivalents are intended to be encompassed by the following claims.

1. A method for processing lignocellulosic material, comprising the steps of: placing a sample of lignocellulosic material in a reactor; adding to said reactor a yeast or fungus at a first temperature and a first pH to give a first mixture; adding to said first mixture a thermophilic microorganism and at least one enzyme at a second temperature and a second pH to give a second mixture; and allowing the second mixture to age for a period of time to give a third mixture; wherein said third mixture comprises a liquid product and a solid product; and said liquid product comprises ethanol.

2. The method of claim 1, further comprising the step of recovering the ethanol.

3. The method of claim 1, wherein both a yeast and a fungus are added.

4. The method of claim 3, wherein at least one enzyme is a cellulolytic enzyme selected from the group consisting of a cellulase, endoglucanase, cellobiohydrolase, and beta-glucosidase.

5. The method of claim 1, further comprising treating the lignocellulosic material with an effective amount of at least one enzyme selected from the group consisting of a hemicellulase, esterase, protease, laccase, and peroxidase.

6. The method of claim 1, wherein said second temperature is above 45° C.

7. (canceled)

8. The method of claim 1, wherein the first pH is about 5.

9. The method of claim 1, wherein the second pH is between 5-6.

10. The method of claim 1, wherein the second pH is between 6-7.

11. The method of claim 1, wherein the second pH is greater than 6.

12-19. (canceled)

20. A method for converting lignocellulosic biomass material into ethanol, the method comprising the steps of:
(i) preparing in a reaction vessel an aqueous slurry of said biomass material;
(ii) adding to said reaction vessel a yeast or fungus resulting in partial separation of the biomass material into cellulose, hemicellulose and lignin;
(iii) adding to said reaction vessel a thermophilic microorganism and at least one enzyme;
(iv) heating for a period of time said reaction vessel to give a mixture;
wherein said mixture comprises a liquid product and a solid product; and said liquid product comprises ethanol.

21. The method of claim 20, wherein the treatment of step (iii) is an anaerobic fermentation process.

22. (canceled)

23. The method of claim 20, wherein the steps are performed as a batch process in a closed, pressurizable reaction vessel having a free volume for containing oxygen-containing gas or water vapor with or without additional gasses.

24. The method of claim 20, wherein the steps are performed as a batch process in a closed, pressurizable reaction vessel with recirculation of the reaction mixture.

25. The method of claim 20, wherein the steps are performed as a continuous process in an essentially tubular reactor.

26. The method of claim 20, wherein step (iii) is performed at a temperature of about 55° C.

27. The method of claim 20, wherein step (iii) is performed at a temperature of greater than 100° C.

28. The method of claim 20, wherein said lignocellulosic material contains, on a dry basis, at least about 20% (w/w) cellulose, at least about 10% (w/w) hemicellulose, and at least about 10% (w/w) lignin.

29. The method of claim 20, wherein said lignocellulosic material is selected from the group consisting of grass, switch grass, cord grass, rye grass, reed canary grass, miscanthus, sugar-processing residues, sugar cane bagasse, agricultural wastes, rice straw, rice hulls, barley straw, corn cobs, cereal straw, wheat straw, canola straw, oat straw, oat hulls, corn fiber, stover, soybean stover, corn stover, forestry wastes, recycled wood pulp fiber, sawdust, hardwood, and softwood.

30-33. (canceled)

34. The method of claim 20, wherein the yeast is selected from the group consisting of Ascomycota, Basidiomycota or Saccharomycetales.

35. The method of claim 34, wherein the yeast is resistant to inhibitors.

36. The method of claim 35, wherein the yeast is genetically engineered or naturally capable of metabolizing the inhibitors.

37. The method of claim 20, wherein the thermophilic microorganism is a species of the genera Thermoanaerobacterium, Thermoanaerobacter, Clostridium, Geobacillus, Saccharococcus, Paenibacillus, Bacillus, or Anoxybacillus.

38. The method of claim 37, wherein the thermophilic microorganism is a bacterium selected from the group consisting of: Thermoanaerobacterium thermosulfurigenes, Thermoanaerobacterium aotearoense, Thermoanaerobacterium polysaccharolyticum, Thermoanaerobacterium zeae, Thermoanaerobacterium xylanolyticum, Thermoanaerobacterium saccharolyticum, Thermoanaerobium brockii, Thermoanaerobacterium thermosaccharolyticum, Thermoanaerobacter thermohydrosulfuricus, Thermoanaerobacter ethanolicus, Thermoanaerobacter brocki, Clostridium thermocellum, Geobacillus thermoglucosidasius, Geobacillus stearothermophilus, Saccharococcus caldoxylosilyticus, Saccharoccus thermophilus, Paenibacillus campinasensis, Bacillus flavothermus, Anoxybacillus kamchatkensis, and Anoxybacillus gonensis.

39. The method of claim 20, wherein the fungus is selected from the group consisting of Chytridiomycota, Blastocladiomycota, Neocallimastigomycota, Zygomycota, Glomeromycota, Ascomycota, Basidiomycota, and T. reesei Rut 30.

40. The method of claim 20, wherein step (ii) comprises adding to said reaction vessel yeast and fungus.

41-45. (canceled)
